(19) United States
(12) Patent Application Publication
(10) Pub. No.: US 2026/0345667 A1
(43) Pub. Date: Nov. 5, 2026

(54) COMPOSITION FOR EQUINE HEALTH, USE AND METHOD

(71) Applicant: Patrick Musitano, Hamilton (CA)

(72) Inventor: Patrick Musitano, Hamilton (CA)

(21) Appl. No.: 19/393,136

(22) Filed: Nov. 18, 2025

Related U.S. Application Data
(60) Provisional application No. 63/721,778, filed on Nov. 18, 2024.

(51) Int. Cl. A61K 31/728; A61P 19/02; A61P 19/04; A61P 29/00

(52) U.S. Cl. CPC A61K 31/728; A61P 19/02; A61P 19/04; A61P 29/00

(57) ABSTRACT

Compositions for and methods of inhibiting, ameliorating and/or treating symptoms of diseases and/or disorders related to connective tissue damage in one or more joints of a horse are described herein. Uses of the compositions described herein are also described.

### RELATED APPLICATIONS

The present application claims priority to U.S. Provisional Patent Application No. 63/721,778 entitled “COMPOSITION FOR EQUINE HEALTH, USE AND METHOD” filed on Nov. 18, 2024, the entire contents of which are hereby incorporated by reference in their entirety.

### TECHNICAL FIELD

The invention relates to the field of treating symptoms of diseases and/or disorders related to connective tissue damage, and more specifically to treating symptoms of diseases and/or disorders related to connective tissue damage in one or more joints of a horse.

### BACKGROUND

Hyaluronan injections, also referred to as hyaluronic acid (HA) injections, are widely used in equine veterinary medicine to support joint health and manage joint disease.

HA is a naturally occurring component of synovial fluid in joints. Hyaluronan provides synovial fluid with viscoelastic properties that are essential for lubricating joints and absorbing shock.

HA injections are a common treatment for horses with arthritis to improve joint health, increase lubrication, and reduce pain. HA can be administered intravenously or intra-articularly directly into the joint. While intra-articular injections may provide immediate benefits, they carry a higher risk of side effects like joint infection or a temporary flare-up of lameness. Also, repeated intra-articular injections are associated with an increased risk of flare reactions, also known as “joint flares”. Joint flares are reactive synovitis caused by inflammation soon after a joint infection.

A joint flare can result in lameness, severe joint effusions, and marked changes in synovial fluid protein content. While “joint flares” can mimic symptoms of a septic joint, prognosis and treatment are quite different. “Joint flares” typically resolve in 1-3 days with only rest and anti-inflammatories.

Accordingly, there is a need for new methods of improving joint health of horses that avoid risks such as joint flares.

### SUMMARY

In accordance with a broad aspect, methods of inhibiting, ameliorating and/or treating symptoms of diseases and/or disorders related to connective tissue damage in one or more joints of a horse are described herein. The methods include administering a therapeutically effective amount of a composition comprising hyaluronic acid (HA) to the horse, the HA having a weight average molecular weight greater than about 1000000 Da.

In accordance with another broad aspect, methods of inhibiting, ameliorating and/or treating symptoms of diseases and/or disorders related to inflammation in one or more joints of a horse are described herein. The methods include administering a therapeutically effective amount of a composition comprising hyaluronic acid (HA) to the horse, the HA having a weight average molecular weight greater than about 1000000 Da.

In at least one embodiment of a method, the HA has a weight average molecular weight in a range of about 1000000 Da to about 3000000 Da.

In at least one embodiment of a method, the HA has a weight average molecular weight of about 2000000 Da.

In at least one embodiment of a method, the HA has a number average molecular weight in a range of about 1 Da to about 1000 Da.

In at least one embodiment of a method, the HA has a number average molecular weight in a range of about 400 Da to about 600 Da.

In at least one embodiment of a method, the HA has a number average molecular weight of about 500 Da.

In at least one embodiment of a method, administering the composition includes administering the composition in a dose having a volume in a range of about 5 mL to about 50 mL.

In at least one embodiment of a method, administering the composition includes administering the composition in a dose having a volume in a range of about 5 mL to about 15 mL.

In at least one embodiment of a method, administering the composition includes administering the composition with a frequency about once per week for a duration of more than one week.

In at least one embodiment of a method, the symptoms of diseases and/or disorders related to connective tissue damage in one or more joints of a horse are lower suspensory ligament inflammation.

In accordance with another broad aspect, uses of a composition for inhibiting, ameliorating and/or treating symptoms of diseases and/or disorders related to connective tissue damage in one or more joints of a horse are described herein. The composition includes hyaluronic acid (HA) having a weight average molecular weight greater than about 1000000 Da.

In at least one embodiment of a use, the HA has a weight average molecular weight in a range of about 1000000 Da to about 3000000 Da.

In at least one embodiment of a use, the HA has a weight average molecular weight of about 2000000 Da.

In at least one embodiment of a use, the HA has a number average molecular weight in a range of about 1 Da to about 1000 Da.

In at least one embodiment of a use, the HA has a number average molecular weight in a range of about 400 Da to about 600 Da.

In at least one embodiment of a use, the HA has a number average molecular weight of about 500 Da.

In at least one embodiment of a use, administering the composition includes administering the composition in a dose having a volume in a range of about 5 mL to about 50 mL.

In at least one embodiment of a use, administering the composition includes administering the composition in a dose having a volume in a range of about 5 mL to about 15 mL.

In at least one embodiment of a use, administering the composition includes administering the composition with a frequency about once per week for a duration of more than one week.

In at least one embodiment of a use, the symptoms of diseases and/or disorders related to connective tissue damage in one or more joints of a horse are lower suspensory ligament inflammation.

### BRIEF DESCRIPTION OF THE DRAWINGS

For a better understanding of the various embodiments described herein, and to show more clearly how these various embodiments may be carried into effect, reference will be made, by way of example, to the accompanying drawing. The drawing is not intended to limit the scope of the teachings described herein.

FIG. 1 shows a response plot and a distribution plot of the molecular weights of a composition including hyaluronic acid, according to at least one embodiment described herein.

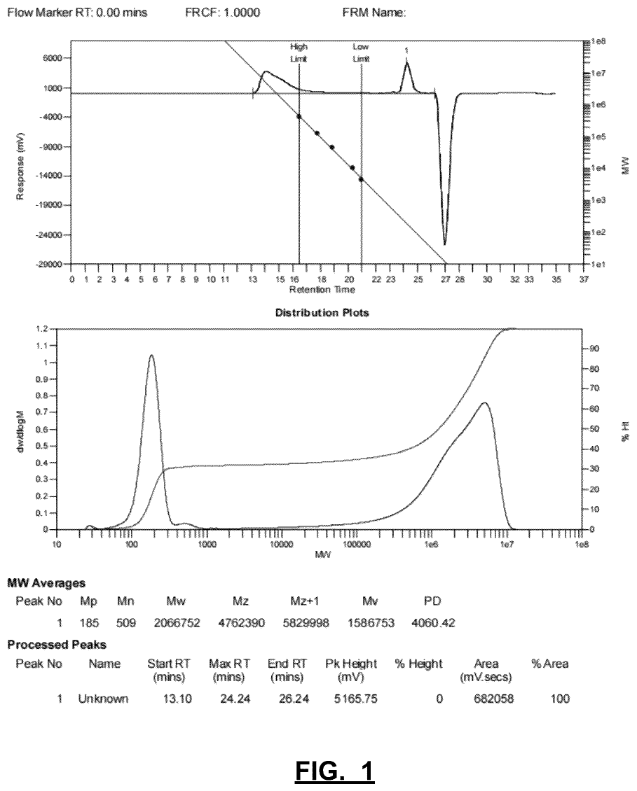

FIG. 2 shows a response plot and a distribution plot of the molecular weights of a composition including hyaluronic acid, according to at least one embodiment described herein.

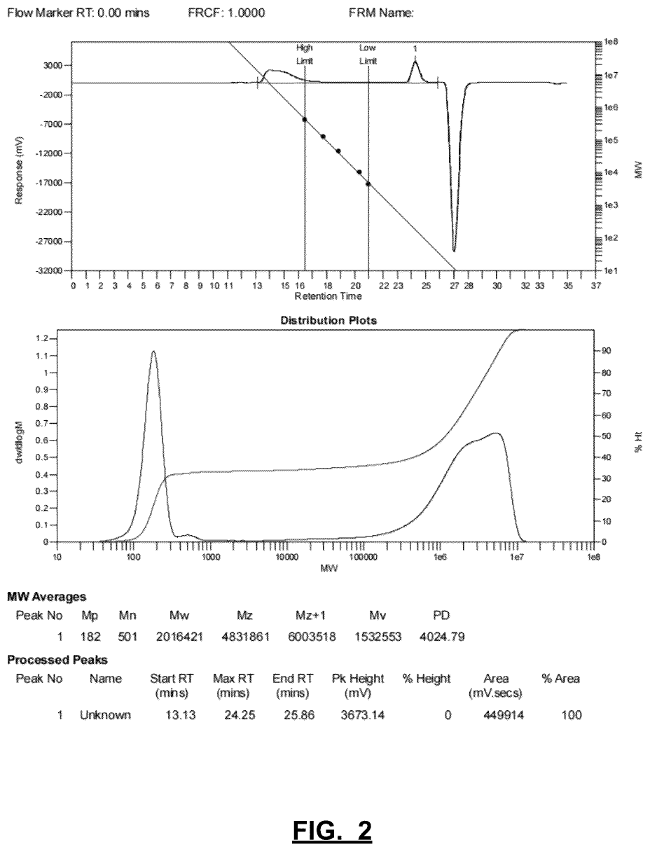

FIGS. 3A and 3B are photographs of a limb of a horse before (A) and after (B) being treated for inflammation with a composition and methods according to at least one embodiment described herein.

FIGS. 4A and 4B are photographs of a limb of another horse before (A) and after (B) being treated for inflammation with a composition and methods according to at least one embodiment described herein.

FIGS. 5A and 5B are photographs of a limb of another horse before (A) and after (B) being treated for inflammation with a composition and methods according to at least one embodiment described herein.

### DETAILED DESCRIPTION

Various compositions and methods will be described below to provide an example of one or more embodiments. No embodiment described below limits any claimed embodiment and any claimed embodiment may cover compositions or methods that differ from those described below. The claimed embodiments are not limited to compositions and methods having all of the features of any one composition or method described below or to features common to multiple or all of the compositions and methods described below. It is possible that a composition or method described below is not an embodiment of any claimed embodiment. Any embodiment disclosed below that is not claimed in this document may be the subject matter of another protective instrument, for example, a continuing patent application, and the applicants, inventors or owners do not intend to abandon, disclaim or dedicate to the public any such embodiment by its disclosure in this document.

As used herein, the term “hyaluronan” of “hyaluronic acid” generally refers to an anionic, non-sulfated glycosaminoglycan distributed widely throughout connective, epithelial, and neural tissues having the following chemical structure:

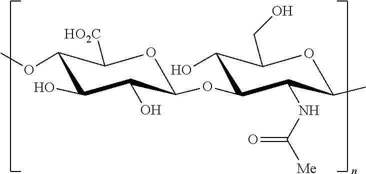

As used herein, Mp refers to molecular weight of the peak maxima.

As used herein, Mn refers to the number average molecular weight.

As used herein, Mw refers to the weight average molecular weight.

As used herein, Mz refers to the Z average molecular weight.

As used herein, Mz+1 refers to the Z+1 average molecular weight.

As used herein, Polydispersity (PD) or polydispersity index refers to the ratio of Mw to Mn.

As used herein, a “therapeutically effective amount” refers to an amount effective, at dosages and for periods of time necessary, to achieve the desired effect.

In general, the compositions and methods described herein are directed to compositions and methods related to a therapy that inhibits, ameliorates and/or treats symptoms of diseases and/or disorders related to connective tissue damage in the joints of horses and do so without causing joint flares, a common side effect of prior art compositions and methods for therapies that inhibits, ameliorates and/or treats symptoms of diseases and/or disorders related to connective tissue damage in the joints of horses.

In at least one embodiment, the connective tissue damage may include but is not limited to arthritic disease, osteoarthritis, rheumatoid arthritis, osterochondrosis dessicans, cartilage damage, joint injury, joint inflammation, joint synovitis, degenerative joint disease (DJD), post-surgical DJD, traumatic injury, fracture, tendon damage, ligament damage, skeletal damage, musculoskeletal damage, fiber damage, adipose tissue damage, small intestine tissue damage, blood cell damage, and plasma damage.

In general, the present document is directed to compositions and methods for improving treatment of arthritis and/or other inflammation and/or disfunction in the joints of horses.

In general, the compositions and methods described herein are directed to compositions and methods for improving treatment of arthritis and/or other inflammation and/or disfunction in the joints of horses and do so without causing joint flares, a common side effect of prior art compositions and methods for improving treatment of arthritis and/or other inflammation and/or disfunction in the joints of horses.

In general, the present document is also directed to compositions and methods related to a therapy that inhibits, ameliorates and/or treats symptoms of diseases and/or disorders related to inflammation, as applied to a wide spectrum of diseases and disorders, through administration of one or more compounds comprising hyaluronic acid.

In general, the compositions and methods described herein are directed to compositions and methods related to a therapy that inhibits, ameliorates and/or treats symptoms of diseases and/or disorders related to inflammation in the joints of horses and do so without causing joint flares, a common side effect of prior art compositions and methods for therapies that inhibits, ameliorates and/or treats symptoms of diseases and/or disorders related to inflammation in the joints of horses.

In general, the present document is directed to compositions and methods related to a therapy that inhibits, ameliorates and/or treats symptoms of diseases and/or disorders and/or adverse effects related to arthritis in the joints of horses.

In general, the compositions and methods described herein are directed to compositions and methods for improving treatment of arthritis in the joints of horses and do so without causing joint flares, a common side effect of prior art compositions and methods for improving treatment of arthritis and/or other inflammation and/or disfunction in the joints of horses.

In general, the present document is also directed to compositions and methods related to a therapy that inhibits, ameliorates and/or treats symptoms of diseases and/or disorders related to connective tissue damage in the joints of horses, as applied to a wide spectrum of diseases and disorders, through administration of one or more compounds comprising hyaluronic acid.

The treatment of connective tissue afflictions can be problematic. A simple decrease in the stress to which the connective tissue is subjected is often not an option, especially in the case of athletes and animals such as racehorses. Herein, the term “racehorse” is used to describe horses that are specifically trained for racing, including but not limited to flat racing (e.g., turf, all-weather, barrel, etc.), jump racing and harness racing. Consequently, treatment of racehorses is often directed at controlling the symptoms of the afflictions and not their causes, regardless of the stage of the degenerative process.

Racehorses that are of an advanced age (i.e., horses no longer in their peak performance years, or generally older than about 4 or 5 years old for flat racers, or older than about 10 years for jump racers) can have a particularly difficult time being treated for connective tissue afflictions.

In at least one embodiment, the present document is also directed to compositions and methods related to treatment of lower suspensory ligament inflammation.

In at least one embodiment, the present document is also directed to compositions and methods related to treatment of lameness in horses. Herein, the term lameness refers to an abnormal gait or stance caused by a physical or functional disorder in the horse's limbs, joints, or muscles, often due to pain but also from neurological or mechanical issues. Lameness can also refer to a general lack of physical ability or a weak, unsatisfactory quality in a non-physical sense, such as an excuse.

The compositions described herein include hyaluronic acid (HA), or an acceptable salt thereof. For example, without limiting the foregoing, the compositions may include a hyaluronic sodium salt sold by Stanford Chemicals under Product Name HA 5229.

In at least one embodiment, the compositions described herein include may also include a phosphate buffer solution (PBS).

In at least one embodiment, the PBS is prepared according to the following formula: Sodium Phosphate, Monobasic x 1H₂O: 0.070 gram; Sodium Phosphate, Dibasic: 0.239 grams; and Sterile Water for Injection (SWFI): qs 100 mL

In at least one embodiment, the composition may have the following formula: HA: 1.1 grams; PBS: 10 mL; normal saline for injection (NS): qs 100 mL.

In at least one embodiment, the composition is sterilized before injection.

In at least one embodiment, the composition has the specifications shown in Table 1, below.

TABLE 1
Specifications of HA composition
Product Name: HA5229 Sodium Hyaluronate (HA-EP1.8-SC)
Grade: Pure Injection-Grade Test Method: EP11.0

| | |
|---|---|
| Test Items | Specifications |

Physical & Chemical Test

| 1 | Appearance | White or almost white, hygroscopic powder | |
|---|---|---|---|
| | | or fibrous aggregate | |
| 2 | Infrared absorption | Positive | |
| 3 | Reaction of sodium | Positive | |
| 4 | Appearance of solution | Clear,A_{600 nm} ≤ 0.01 | |
| 5 | pH | 5.0~8.5 | |
| 6 | Intrinsic viscosity | 1.44-2.12 | m³/kg |

Purity Test

| 7 | Nucleic acids | A_{260 nm} ≤0.5 | |
|---|---|---|---|
| 8 | Protein | ≤0.1% | |
| 9 | Chlorides | ≤0.5% | |
| 10 | Loss on drying | ≤20% | |
| 11 | Iron | ≤80 | ppm |
| 12 | Residual | ≤5000 | ppm |
| | solvents(Ethanol) | | |

| | | |
|---|---|---|
| 13 | Assay | 95.0%~105.0% |

Bacterial endotoxins test

| | | | |
|---|---|---|---|
| 14 | Bacterial endotoxins | ≤0.05 | IU/mg |

Microbiological Purity

| | | | |
|---|---|---|---|
| 15 | Total bacterial count | ≤100 | CFU/g |
| 16 | Yeasts & mold | ≤20 | CFU/g |

| | | |
|---|---|---|
| 17 | E. coli | Negative |
| 18 | Staph | Negative |
| 19 | Pseudomonas | Negative |
| | aeruginosa | |

In at least one embodiment, the HA has a weight average molecular weight greater than about 1000000 Da, or greater than about 2000000 Da, or greater than about 3000000 Da, or in a range of about 1000000 Da to about 3000000 Da, or in a range of about 1000000 to about 2000000 Da, or in a range of about 2000000 Da to about 3000000 Da, or of about 20000000 Da.

In at least one embodiment, the HA has a number average molecular weight in a range of about 1 Da to about 1000 Da, or in a range of about 200 Da to about 800 Da, or in a range of about 400 Da to about 600 Da, or of about 500 Da. As noted above, the number average molecular weight represents the average molecular weight of a polymer sample. It is calculated by dividing the total weight of all molecules by the total number of molecules in the sample.

In at least one embodiment, the HA has a polydispersity of about 4000.

In at least one embodiment, the composition is administered to a horse, the composition having a HA concentration in a range of about 1 mg/mL to about 20 mg/mL, or in a range of about 1 mg/mL to about 15 mg/mL, or in a range of about 5 mg/mL to about 20 mg/mL, or in a range of about 5 mg/mL to about 15 mg/mL, or in a range of about 8 mg/mL to about 13 mg/mL, or in a range of about 10 mg/mL to about 12 mg/mL, or of about 11 mg/mL

In at least one embodiment, the composition is administered via intra-articular (IA) injection to the horse. For example, IA injection may include injection directly into the affected joint. In at least one embodiment, IA injection may provide targeted relief and maximum drug concentration in the joint.

In at least one embodiment, the composition is administered via intravenous (IV) injection to the horse: For example, IV injection may include administration into the vein. IV injection may have a systemic anti-inflammatory effect.

In at least one embodiment, the composition may be administered via IV injection to the jugular vein of the horse.

The methods described herein will generally not be limited to any particular dosage form or route of administration. Although IA and IV will generally be most convenient, the methods are compatible with other dosage forms or routes of administration as well.

In at least one embodiment, the composition may be administered in doses having a volume in a range of about 1 mL to about 100 mL, or in a range of about 5 mL to about 50 mL, or in a range of about 5 mL to about 20 mL, or in a range of about 5 mL to about 15 mL, or of about 5 mL, or 6 mL, or 7 mL, or 8 mL, or 9 mL, or 10 ml, or 11 mL, or 12 mL, or 13 mL, or 14 mL, or 15 mL.

In at least one embodiment, the composition may be administered in doses having a volume noted above in a frequency of about once per week, or about once per two weeks, or about once per three weeks, or about once per four weeks, or about every day, or about once every two days, or about once every three days, or about once every four days, or about once every five days, or about once every six days, or about once every seven days, or about once every eight days, or about once every nine days, or about once every ten days.

Therapeutic compositions typically should be sterile and stable under the conditions of manufacture and storage.

### Examples

In one example, a veterinarian administered the composition noted above in Table 1 to numerous patients, including their own racehorse. Each administration involved the IV injection of 5 mL of the noted composition into the right jugular vein of the horse.

The veterinarian noted that most horses showed improvement after the second treatment, but some showed improvement after the initial treatment. In terms of the improvement, the treatment caused the horses to stay sounder for longer, allowing IV injections to be conducted less frequently.

The veterinarian also noted that the IV injection of the composition noted in Table 1 provided for injections to be done less frequently, without deleterious impacts on horse comfort, than other HA injections they'd used in the past.

In another example, 3 mL doses of 18 mg/mL of HA with a relatively high molecular weight (about 5000000 Da) was injected into horses and observed to produce a reaction (e.g., flare) about 24 hours after injection in the horses.

In contrast, 5 mL doses of 11 mg/mL of HA with a lower molecular weight of approximately 2000000 Da was injected into 20 joints involving five horses. Observations were made at 30 hrs, 48 hrs and 72 hrs post injection. There were no visible reactions noted. Photographs of the hocks of each subject were captured pre- and post-injection, a few of which are provided herein. The hocks were chosen in particular due to the tendency for this joint in particular to show flares. The results are tabulated below.

TABLE 2
Summary of Results after HA Injection

| | | | Observa- | Observa- | Observa- |
|---|---|---|---|---|---|
| | Age, sex, | Joints | tions | tions | tions |
| | breed | injected | 30 hrs. | 48 hrs. | 72 hrs. |
| 1. | 12 yrs, female, | L + R carpus | No flares, | No flares | No flares |
| | Quarter horse | L + R hocks | hind legs | | |
| | | RF fetlock | slightly | | |
| | | LH fetlock | stocked up | | |
| | | | (normal for | | |
| | | | this mare) | | |
| 2. | 14 yrs, female, | L + R carpus | No flares | No flares | No flares |
| | Quarter horse | L + R hocks | | | |
| 3. | 27 yrs, male, | L + R hocks | No flares | No flares | No flares |
| | Quarter horse | | | | |
| 4. | 13 yrs, male, | L + R hocks | No flares | No flares | No flares |
| | Quarter horse | L + R front | | | |
| | | fetlocks | | | |
| 5. | 15 yrs, female, | L + R carpus | No flares | No flares | No flares |
| | Quarter horse | L + R hocks | | | |

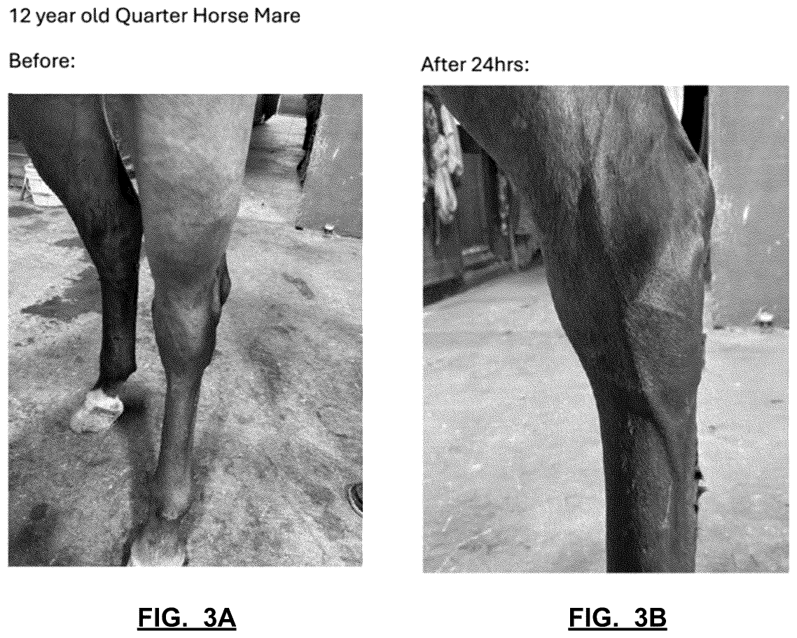

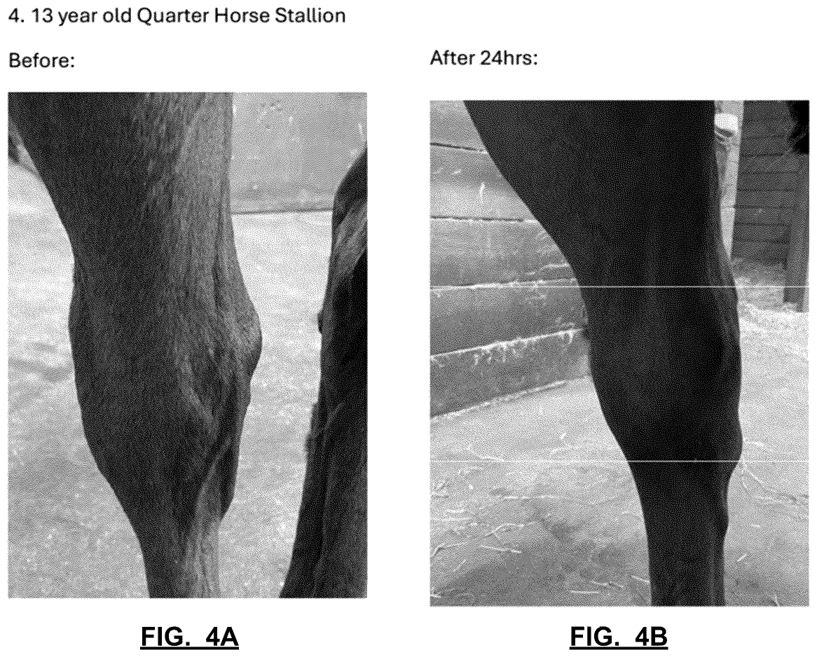

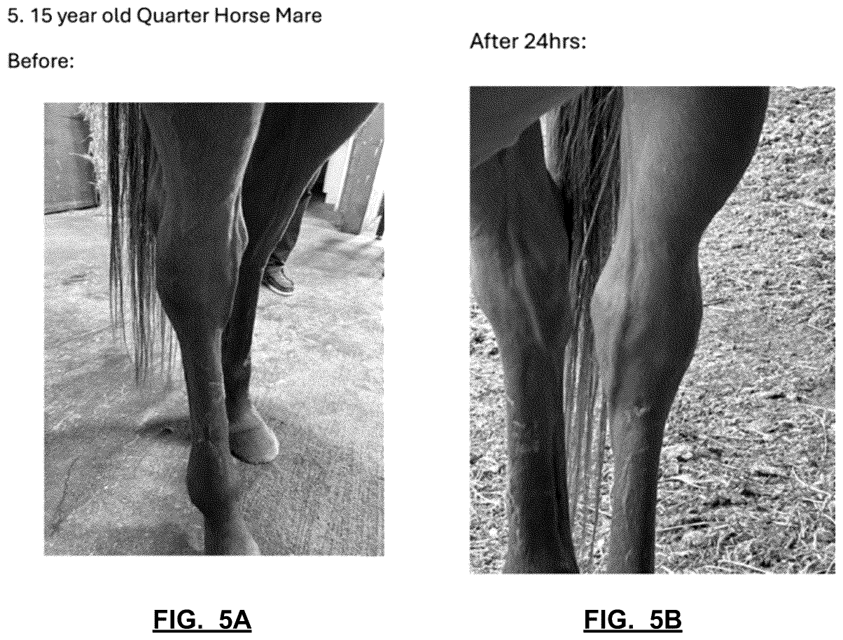

Images of the legs of the horses noted in rows 1, 4 and 5 of Table 1 are shown in FIGS. 3A and 3B, 4A and 4B and 5A and 5B, respectively.

None of these horses were previously treated with HA. The absence of flares in these horses with the lower molecular weight product (2000000 Da) was particularly surprising, given the usage of higher molecular weight HA in other studies. It should also be noted that no prophylactic corticosteroids or NSAIDs were given at the time of injection.

In additional studies, it was determined that the lower molecular weight product (2000000 Da) was most efficacious.

Table 3 shows a summary of observations of compositions with various molecular weights of HA.

TABLE 3
Summary of observations of compositions with
various molecular weights of HA
Formulation Development and Observations

| | Approx. Molecular Weight | Key Observations/ |
|---|---|---|
| Formulation | (Da) | Outcomes |
| 18 mg/mL | ~5,000,000 | High IA efficacy; flares |
| | | common; unsuitable |
| | | viscosity for IV use. |
| 15 mg/mL | ~4,000,000 | Improved tolerability; still |
| | | viscous; occasional flares. |
| 13 mg/mL | ~3,000,000 | Fewer flares; moderate |
| | | inflammation. |
| 11 mg/mL | ~2,000,000 | Optimal viscosity; flare-free; |
| | | IA & IV compatible. |
| 9 mg/mL | ~1,500,000 | Shorter duration of benefit; |
| | | reduced efficacy. |
| 7 mg/mL | ≤1,000,000 | Too thin; limited benefit. |

Table 4 shows a summary of observations of various dosing intervals of the compositions described herein.

TABLE 4
Summary of observations of various dosing intervals
of the compositions described herein
Route and Dosing Interval Optimization

| Interval | Observation Summary |
|---|---|
| Every 2 weeks | Moderate benefit; effects waned before |
| | next dose. |
| Every 3 weeks | Comfort decline at end of cycle. |
| Every other day | No added value; unnecessary cost. |
| Weekly (10 mL IV) | Sustained comfort, no flares. |

Table 5 shows a summary of field testing of the compositions described herein.

TABLE 5
Summary of Field Tests
Multi Visk Development and Field Testing Timeline (2024-2025)

| Date | Study Phase | Summary of Findings |
|---|---|---|
| June 2024 | 18 mg/mL | Mild flares (~9%); |
| | | viscosity too high. |
| July 2024 | 13 mg/mL | Rapid recovery; flares |
| | | dropped to ~6%. |
| November 2024 | 9 mg/mL | No flares; shorter efficacy |
| | | (~1 week). |
| May 2025 | 11 mg/mL | No flares across 20 joints; |
| | | strong 2-3 week effect. |
| June-July 2025 | Trainer | Improved flexibility and |
| | Integration | endurance. |
| August 2025 → | Follow-Up | Continued IA tracking |
| | | planned. |

Tables 6-9 show summaries of specific testing of joints of horses using HA of various molecular weights.

TABLE 6
Summary of Specific Testing of Joints of Horses with 18 mg/mL HA
Phase 1-June 2024 (18 mg/mL)

| | | Joints | | | |
|---|---|---|---|---|---|
| # | Age/Sex/Breed | Injected | 30 h Obs | 48 h Obs | 72 h Obs |
| 1 | 11 F QH | L + R hocks | Mild | Improved | Resolved |
| | | | swelling | | |
| 2 | 13 F QH | L + R fetlocks | No flare | No flare | No flare |
| 3 | 15 M QH | L + R carpi | No flare | No flare | No flare |
| 4 | 16 M TB | L + R hocks | Mild heat | Normal | Resolved |
| 5 | 18 F QH | L + R fetlocks | No flare | No flare | No flare |

TABLE 7
Summary of Specific Testing of Joints of Horses with 13 mg/mL HA
Phase 2-July 2024 (13 mg/mL)

| | | Joints | | | |
|---|---|---|---|---|---|
| # | Age/Sex/Breed | Injected | 30 h Obs | 48 h Obs | 72 h Obs |
| 1 | 10 M QH | Carpi + | No flare | No flare | No flare |
| | | hocks | | | |
| 2 | 17 F WB | L + R fetlocks | Mild | Resolved | Normal |
| | | | swelling | | |
| 3 | 12 M QH | L + R hocks | No flare | No flare | No flare |
| 4 | 15 M QH | L + R carpi | No flare | No flare | No flare |
| 5 | 20 F Paint | L carpus | No flare | No flare | No flare |

TABLE 8
Summary of Specific Testing of Joints of Horses with 9 mg/mL HA
Phase 3-November 2024 (9 mg/mL)

| | | Joints | | | |
|---|---|---|---|---|---|
| # | Age/Sex/Breed | Injected | 30 h Obs | 48 h Obs | 72 h Obs |
| 1 | 8 M QH | L + R hocks | No flare | No flare | No flare |
| 2 | 13 F QH | L + R fetlocks | No flare | No flare | No flare |
| 3 | 15 F QH | L + R carpi | No flare | No flare | No flare |
| 4 | 16 M QH | L + R hocks | No flare | No flare | No flare |

TABLE 9
Summary of Specific Testing of Joints of Horses with 11 mg/mL HA
Phase 4-May 2025 (11 mg/mL)

| | | Joints | | | |
|---|---|---|---|---|---|
| # | Age/Sex/Breed | Injected | 30 h Obs | 48 h Obs | 72 h Obs |
| 1 | 12 F QH | Carpi, hocks, | Mild | No flares | No flares |
| | | RF & LH | stocking-up | | |
| | | fetlocks | | | |
| 2 | 14 F QH | Carpi + | No flares | No flares | No flares |
| | | hocks | | | |
| 3 | 27 M QH | L + R hocks | No flares | No flares | No flares |
| 4 | 13 M QH | Hocks + | No flares | No flares | No flares |
| | | front | | | |
| | | fetlocks | | | |
| 5 | 15 F QH | Carpi + | No flares | No flares | No flares |
| | | hocks | | | |

Table 10 shows a summary of specific testing of joints of horses using HA of various molecular weights at different strengths.

TABLE 10
Summary of Specific Testing of Joints of Horses with 18 mg/mL HA
Trainer Performance Evaluation - Jon Newnham (June-July 2025)

| Phase | Strength | Flare Rate | Feedback | Rating | Notes |
|---|---|---|---|---|---|
| 1 | 18 mg/mL | 9% | Stiff post- | 7/10 | Recovery |
| | (5M) | | injection; | | in 3 days |
| | | | gradual | | |
| | | | improvement | | |
| 2 | 13 mg/mL | 6% | Fast recovery; | 8.5/10 | Full work |
| | (3M) | | smoother | | in 48 h |
| | | | stride | | |
| 3 | 9 mg/mL | 0% | Comfortable | 6/10 | Effect fades |
| | (1.5M) | | but shorter | | day 7 |
| | | | benefit | | |
| 4 | 11 mg/mL | 0% | Unprecedented | 9.5/10 | Peak 2-3 |
| | (2M) | | flexibility; strong | | weeks |
| | | | endurance | | |

As shown in Tables 6-10, several horses that had previously been lame or showing arthritic stiffness demonstrated remarkable improvement after only two IV treatments administered over a two-week period. Horses that were short-striding, resistant, or uncomfortable began moving more freely and returned to regular work sooner than expected. With continued weekly IV therapy, progressive recovery in soft-tissue structures was also observed. The most notable improvement occurred in horses with lower suspensory ligament inflammation—an injury that normally requires 6 to 12 months of rehabilitation. In these cases, the ligaments went from visibly inflamed and highly sensitive to nearly fully resolved within approximately two months, showing reductions in pain, heat, and swelling that far exceeded normal expectations.

The 11 mg/mL (~2000000 Da) formulation provided an optimal balance of viscosity and biological activity, eliminating flare reactions and enabling safe, effective dual-route (IA and IV) use. Veterinary data and trainer feedback aligned consistently, confirming uniform performance and extended therapeutic duration.

In another example, a 11 mg/mL (~2000000 Da) formulation administered by IA once per day resulted in peak efficacy (i.e., no flares, improved flexibility and endurance of the horse) 7-8 days after treatment was initiated. The improved flexibility and endurance of the horse lasted for 5-8 weeks during peak performance season.

In another example, a 11 mg/mL (~2000000 Da) formulation administered by IV in a volume of 10 mL once per week was effective during the performance season and provided for the veterinarian to forgo IA administration and place the horse on an IV-only program. This proved to be not only surprisingly effective but also cost effective.

While the above description provides examples of one or more apparatus, methods, or systems, it will be appreciated that other apparatus, methods, or systems may be within the scope of the claims as interpreted by one of skill in the art.

What is claimed is:

1. A method of inhibiting, ameliorating and/or treating symptoms of diseases and/or disorders related to connective tissue damage in one or more joints of a horse, the method comprising:
administering a therapeutically effective amount of a composition comprising hyaluronic acid (HA) to the horse, the HA having a weight average molecular weight greater than about 1000000 Da.

2. The method of claim 1, wherein the HA has a weight average molecular weight in a range of about 1000000 Da to about 3000000 Da.

3. The method of claim 1, wherein the HA has a weight average molecular weight of about 2000000 Da.

4. The method of claim 1, wherein the HA has a number average molecular weight in a range of about 1 Da to about 1000 Da.

5. The method of claim 1, wherein the HA has a number average molecular weight in a range of about 400 Da to about 600 Da.

6. The method of claim 1, wherein the HA has a number average molecular weight of about 500 Da.

7. The method of claim 1, wherein administering the composition includes administering the composition in a dose having a volume in a range of about 5 mL to about 50 mL.

8. The method of claim 1, wherein administering the composition includes administering the composition in a dose having a volume in a range of about 5 mL to about 15 mL.

9. The method of claim 1, wherein administering the composition includes administering the composition with a frequency about once per week for a duration of more than one week.

10. The method of claim 1, wherein the symptoms of diseases and/or disorders related to connective tissue damage in one or more joints of a horse are lower suspensory ligament inflammation.

11. A method of inhibiting, ameliorating and/or treating symptoms of diseases and/or disorders related to inflammation in one or more joints of a horse, the method comprising:
administering a therapeutically effective amount of a composition comprising hyaluronic acid (HA) to the horse, the HA having a weight average molecular weight greater than about 1000000 Da.

12. The method of claim 11, wherein the HA has a weight average molecular weight in a range of about 1000000 Da to about 3000000 Da.

13. The method of claim 11, wherein the HA has a weight average molecular weight of about 2000000 Da.

14. The method of claim 11, wherein the HA has a number average molecular weight in a range of about 1 Da to about 1000 Da.

15. The method of claim 11, wherein the HA has a number average molecular weight in a range of about 400 Da to about 600 Da.

16. The method of claim 11, wherein the HA has a number average molecular weight of about 500 Da.

17. The method of claim 11, wherein administering the composition includes administering the composition in a dose having a volume in a range of about 5 mL to about 50 mL.

18. The method of claim 11, wherein administering the composition includes administering the composition in a dose having a volume in a range of about 5 mL to about 15 mL.

19. The method of claim 11, wherein administering the composition includes administering the composition with a frequency about once per week for a duration of more than one week.

20. The method of claim 11, wherein the symptoms of diseases and/or disorders related to connective tissue damage in one or more joints of a horse are lower suspensory ligament inflammation.
